(19) United States
(12) Patent Application Publication
(10) Pub. No.: US 2013/0102387 A1
(43) Pub. Date: Apr. 25, 2013

(54) CALCULATING METABOLIC EQUIVALENCE WITH A COMPUTING DEVICE

(71) Applicants: Emad Barsoum, Bellevue, WA (US); Ron Forbes, Seattle, WA (US); Tommer Leyvand, Seattle, WA (US); Tim Gerken, Newcastle, WA (US)

(72) Inventors: Emad Barsoum, Bellevue, WA (US); Ron Forbes, Seattle, WA (US); Tommer Leyvand, Seattle, WA (US); Tim Gerken, Newcastle, WA (US)

(73) Assignee: MICROSOFT CORPORATION, Redmond, WA (US)

(21) Appl. No.: 13/279,124

(22) Filed: Oct. 21, 2011

(51) Int. Cl. A63F 9/24; A61B 5/00

(52) U.S. Cl. 463/36; 600/301

(57) ABSTRACT

A method for estimating a metabolic equivalent of task for use with a computing device is provided herein. The method includes receiving input from a capture device of a user; and tracking a position of each of the plurality of joints of the user. The method further includes determining a distance traveled for each of the plurality of joints between a first frame and a second frame; and calculating a horizontal velocity and a vertical velocity for each of the plurality of joints based on the distance traveled and an elapsed time between the first and second frames. The method further includes estimating a value for the metabolic equivalent of task using a metabolic equation including a component for the horizontal velocity and a component for the vertical velocity for each of the plurality of joints; and outputting the value for display.

### BACKGROUND

Computer gaming systems have evolved to include more physically demanding activities, particularly those computer gaming systems equipped with natural input devices such as depth cameras. As such, gaming has become a form of exercise for some users. However, it is difficult for those users to ascertain with precision the rigor of an exercise, such as how many calories a particular exercise has burned. One prior solution can be found in a computer game designed to simulate running. The running game displays to the user a metabolic equivalent of task (MET) for the running activity, which may be used to determine calories burned. However, MET models are task specific, and thus this running game is built on a running-specific MET model, which can only be applied to running. The drawback to a task-specific approach is that many movements in computer gaming are “non-standard activities” and no MET models exist for these activities. Further, custom designing MET models for such activities would be prohibitively expensive and take significant development time. For this reason, most computer games cannot provide a MET value or caloric output estimation for such non-standard activities, frustrating budding computer-based exercisers.

### SUMMARY

A method for estimating a metabolic equivalent of task for use with a computing device is provided herein. The method includes receiving input from a capture device of a user; and tracking a position of each of the plurality of joints of the user. The method further includes determining a distance traveled for each of the plurality of joints between a first frame and a second frame; and calculating a horizontal velocity and a vertical velocity for each of the plurality of joints based on the distance traveled and an elapsed time between the first and second frames. The method further includes estimating a value for the metabolic equivalent of task using a metabolic equation including a component for the horizontal velocity and a component for the vertical velocity for each of the plurality of joints; and outputting the value for display.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 is a perspective view of an example gaming system viewing an observed scene in accordance with an embodiment of the present disclosure.

FIG. 2A schematically shows a human target in an observed scene being modeled with example skeletal data by the gaming system of FIG. 1.

FIG. 2B schematically shows example skeletal data tracked over time by the gaming system of FIG. 1.

FIG. 3 is a flowchart that illustrates an example embodiment of a method for estimating a metabolic equivalent of task using the gaming system of FIG. 1.

FIG. 4 is a flowchart that illustrates an example embodiment of a method for weighing each of a plurality of joints of a user using the gaming system of FIG. 1.

FIG. 5 is a schematic view of a computing system that may be used as the gaming system of FIG. 1.

### DETAILED DESCRIPTION

Aspects of this disclosure will now be described by example and with reference to the illustrated embodiments listed above.

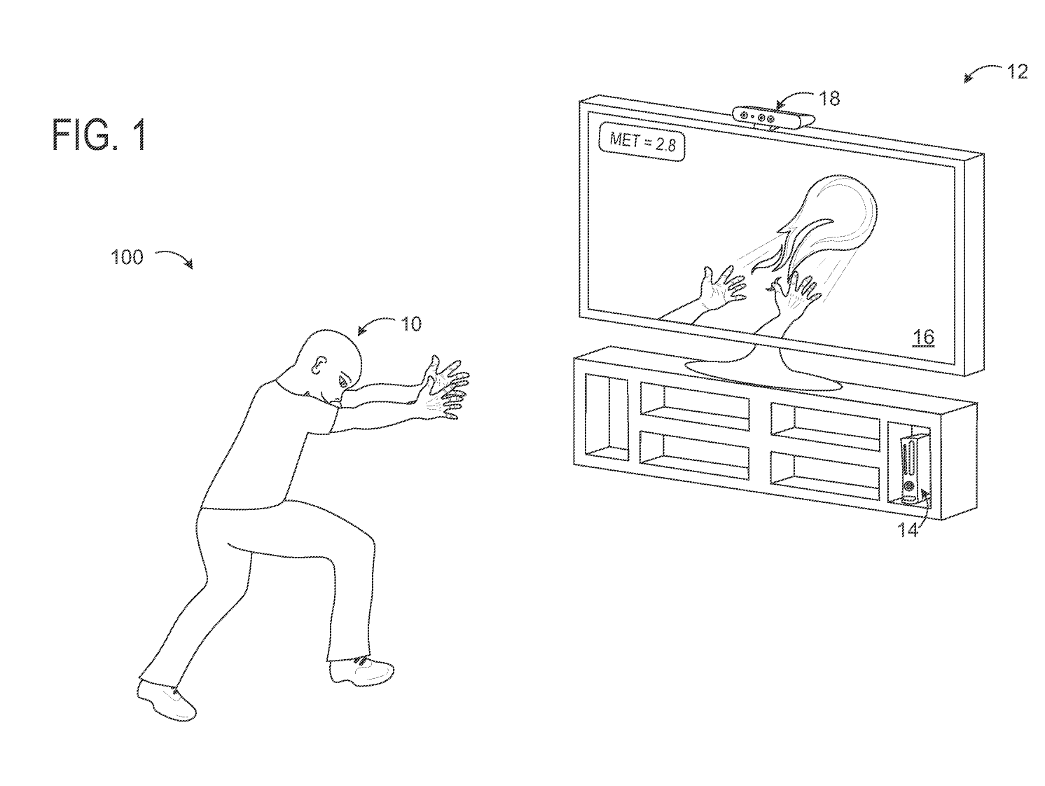

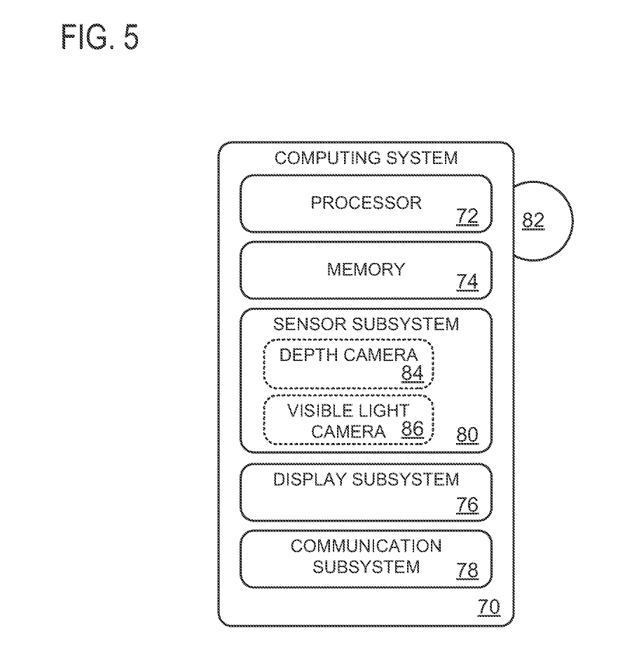

FIG. 1 shows an example 3D interaction space 100 in which user 10 is located. FIG. 1 also shows gaming system 12 which may enable user 10 to interact with a video game. Gaming system 12 may be used to play a variety of different games, play one or more different media types, and/or control or manipulate non-game applications and/or operating systems. Gaming system 12 may include gaming console 14 and display device 16, which may be used to present game visuals to game players. Gaming system 12 is a type of computing device, the details of which will be discussed with respect to FIG. 5.

Turning back to FIG. 1, 3D interaction space 100 may also include a capture device 18 such as a camera, which may be coupled to gaming system 12. Capture device 18, for example, may be a depth camera used to observe 3D interaction space 100 by capturing images. As such, capture device 18 may be used to estimate a metabolic equivalent of task (MET) of user 10 by tracking a position of each of a plurality of joints of user 10. For example, capture device 18 may capture images of the user which may be used to determine a delta distance for each joint, and further, to calculate a velocity of each joint. Further, one or more joints may be weighed differently than other joints to account for various factors such as gravity, user anatomy, user physical ability, degrees of freedom, etc. In this way, user 10 may interact with gaming system, and a value for the MET may be estimated based on the actual movement (or lack thereof) of user 10.

Traditional methods for estimating MET are based on a specific activity or task. One traditional approach involves determining the specific activity the user is engaged in, and outputting an average MET value for that specific activity to a user. This approach does not estimate the MET value based on what the user is actually doing.

Instead, this approach operates on the assumption that a specific activity always has the same MET value regardless of the intensity in which the user performs the specific activity, so that the MET output will be erroneous for most users. Further, this approach does not work for non-standard activities (e.g., non-physical activities) for which there is no average MET value available.

Another traditional approach estimates the MET value based on a velocity detected for a segment of the user's body (e.g., the user's legs). However, this approach also assumes a particular activity, and uses an activity specific MET model to estimate the MET value based on that particular activity. Therefore, this approach is also activity-specific and hence not generic enough to estimate an MET value for non-standard activities.

The present disclosure overcomes at least some of these challenges by estimating the MET value for a user without regard to a type of activity that user 10 is performing. Since the MET value is estimated without limiting the MET value to a particular activity, a more accurate MET value that reflects an intensity of user 10 interacting with gaming system 12 can be estimated. In other words, the MET value for the user is estimated without gaming system 12 assuming or determining what activity the user is performing. Therefore, user 10 may perform virtually any activity and gaming system 12 can estimate the MET value by tracking the movement of user 10 in real-time.

For example, user 10 may interact with gaming system 12 by playing a magic game, a combat game, a boxing game, a dancing game, a racing game, etc., and the user's MET may be estimated without assuming the user is casting a spell, fighting enemies, boxing, dancing, or racing. Further, user 10 may interact with gaming system 12 by watching a movie, interacting with various applications, etc. Such examples may be referred to herein as non-standard activities, yet since the methods described herein estimate MET without assuming a particular activity, the MET value can be estimated for even non-standard activities that may be associated with conceivably less intensity.

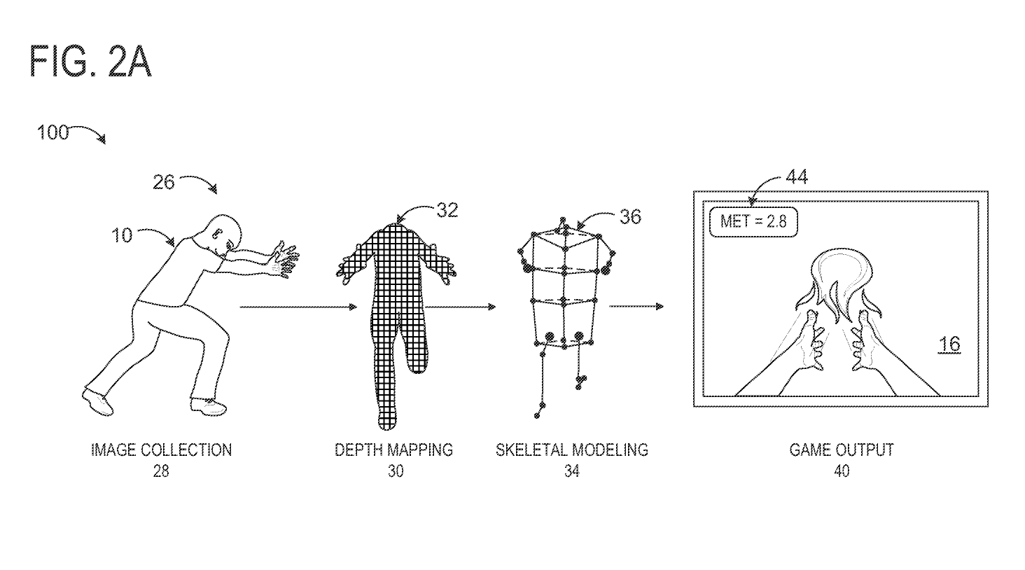

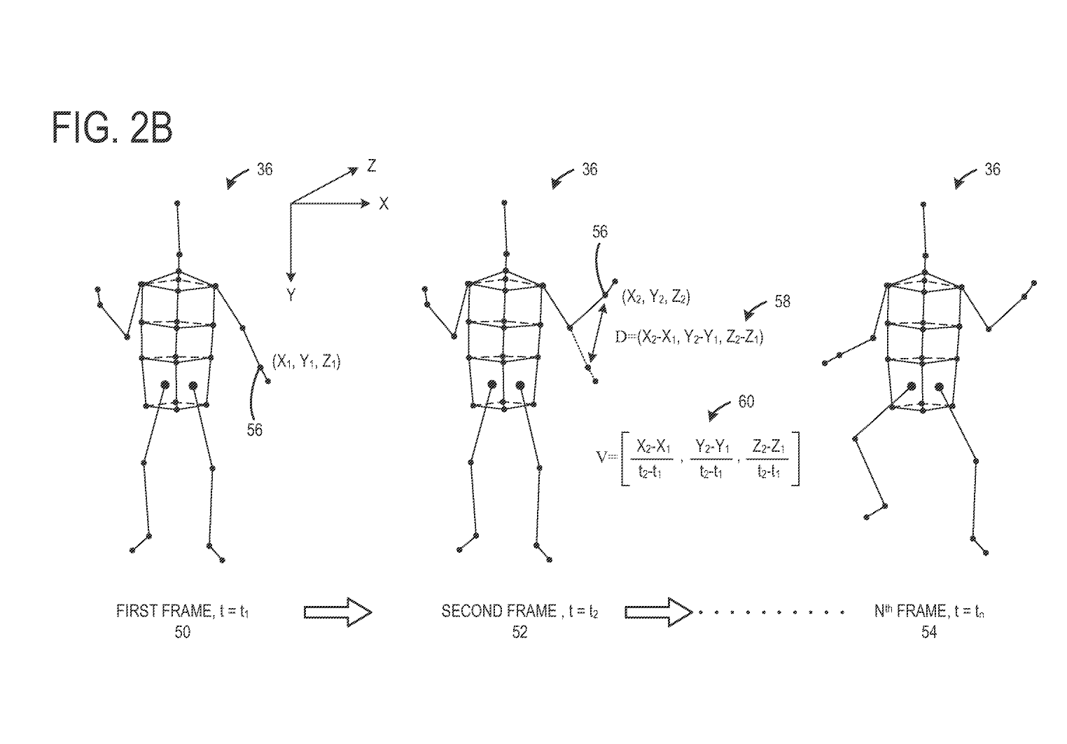

FIG. 2A shows a simplified processing pipeline 26 in which game player 10 in 3D interaction space 100 is modeled as a virtual skeleton 36 that can serve as a control input for controlling various aspects of a game, application, and/or operating system. FIG. 2A shows four stages of the processing pipeline 26: image collection 28, depth mapping 30, skeletal modeling 34, and game output 40. It will be appreciated that a processing pipeline may include additional steps and/or alternative steps than those depicted in FIG. 2A without departing from the scope of this disclosure.

During image collection 28, game player 10 and the rest of 3D interaction space 100 may be imaged by a capture device such as depth camera 18. In particular, the depth camera is used to track a position of each of a plurality of joints of a user (e.g., game player 10). During image collection 28, the depth camera may determine, for each pixel, the depth of a surface in the observed scene relative to the depth camera. Virtually any depth finding technology may be used without departing from the scope of this disclosure. Example depth finding technologies are discussed in more detail with reference to FIG. 5.

During depth mapping 30, the depth information determined for each pixel may be used to generate a depth map 32. Such a depth map may take the form of virtually any suitable data structure, including but not limited to a depth image buffer that includes a depth value for each pixel of the observed scene. In FIG. 2A, depth map 32 is schematically illustrated as a pixelated grid of the silhouette of game player 10. This illustration is for simplicity of understanding, not technical accuracy. It is to be understood that a depth map generally includes depth information for all pixels, not just pixels that image the game player 10. Depth mapping may be performed by the depth camera or the computing system, or the depth camera and the computing system may cooperate to perform the depth mapping.

During skeletal modeling 34, one or more depth images (e.g., depth map 32) of the 3D interaction space including a computer user (e.g., game player 10) are obtained from the depth camera. Virtual skeleton 36 may be derived from depth map 32 to provide a machine readable representation of game player 10. In other words, virtual skeleton 36 is derived from depth map 32 to model game player 10. The virtual skeleton 36 may be derived from the depth map in any suitable manner. In some embodiments, one or more skeletal fitting algorithms may be applied to the depth map. For example, a prior trained collection of models may be used to label each pixel from the depth map as belonging to a particular body part, and virtual skeleton 36 may be fit to the labeled body parts. The present disclosure is compatible with virtually any skeletal modeling technique. In some embodiments, machine learning may be used to derive the virtual skeleton from the depth images.

The virtual skeleton provides a machine readable representation of game player 10 as observed by depth camera 18. The virtual skeleton 36 may include a plurality of joints, each joint corresponding to a portion of the game player. Virtual skeletons in accordance with the present disclosure may include virtually any number of joints, each of which can be associated with virtually any number of parameters (e.g., three dimensional joint position, joint rotation, body posture of corresponding body part (e.g., hand open, hand closed, etc.) etc.). It is to be understood that a virtual skeleton may take the form of a data structure including one or more parameters for each of a plurality of skeletal joints (e.g., a joint matrix including an x position, a y position, a z position, and a rotation for each joint). In some embodiments, other types of virtual skeletons may be used (e.g., a wireframe, a set of shape primitives, etc.).

Skeletal modeling may be performed by the computing system. In particular, a skeletal modeling module may be used to derive a virtual skeleton from the observation information (e.g., depth map 32) received from the one or more sensors (e.g., depth camera 18 of FIG. 1). In some embodiments, the computing system may include a dedicated skeletal modeling module that can be used by a variety of different applications. In this way, each application does not have to independently interpret depth maps as machine readable skeletons. Instead, the individual applications can receive the virtual skeletons in an anticipated data format from the dedicated skeletal modeling module (e.g., via an application programming interface or API). In some embodiments, the dedicated skeletal modeling module may be a remote modeler accessible via a network. In some embodiments, an application may itself perform skeletal modeling.

As introduced above, a value for MET may be estimated by tracking the movements of the game player. It will be appreciated that the skeletal modeling techniques as described above may provide machine readable information including a three-dimensional position of each of a plurality of skeletal joints representing the game player over time. Such data may be used, at least in part, to estimate the MET for the user as described in more detail below.

FIG. 2B shows an example of tracking movements of the game player using skeletal modeling techniques. As described above, the game player may be modeled as virtual skeleton 36. As shown, virtual skeleton 36 (and thus game player 10) may move over time such that one or more joints of the virtual skeleton change in three-dimensional position between a first frame and a second frame, for example. It will be appreciated that to change position, one or more parameters may change. For example, a joint may change a position in an x direction, but may not change in a y and/or z direction. Virtually any change in position is possible without departing from the scope of this disclosure.

As shown in FIG. 2B, a first frame 50 may be followed by a second frame 52, and each frame may include a virtual skeleton 36 that models game player 10 in 3D interaction space 100 as described above. Further, skeletal modeling may proceed for any suitable period of time, for example, to an n^{th} frame 54. It will be appreciated that ‘second frame’ (and likewise, n^{th} frame) as used herein, may refer to a frame that occurs after first frame, wherein after may be for any suitable period of time.

First frame 50 may include virtual skeleton 36 with a left wrist joint 56 determined to have a 3D position of X₁, Y₁, Z₁, as shown. Further, second frame 52 may include virtual skeleton 36 with left wrist joint 56 determined to have a 3D position of X₂, Y₂, Z₂, as shown. Since at least one position parameter of wrist joint 56 has changed between first frame 50 and second frame 52, a distance traveled by joint 56 may be determined. In other words, the distance may be determined based on a change in position of wrist joint 56 between the first and second frames. As shown, the distance may be determined using a formula 58, for example. Further, a velocity for joint 56 may be calculated according to formula 60, for example. As shown, formula 60 may be based on the determined distance and an elapsed time between first frame 50 and second frame 52. Methods for determining the distance traveled by a joint, calculating the velocity of that movement, and other calculations leading to estimating a value for MET are described further below.

Turning back to FIG. 2A, during game output 40, the physical movements of game player 10 as recognized via skeletal modeling 34 are used to control aspects of a game, application, or operating system. Further, such interactions may be measured by estimating the MET value from the detected positions of each of a plurality of joints of the virtual skeleton representing game player 10. In the illustrated scenario, game player 10 is playing a fantasy themed game and has performed a spell throwing gesture. The movements associated with performing the spell throwing gesture may be tracked such that the value for MET can be estimated. As shown, the estimated value for MET (indicated generally at 44) may be displayed on display device 16.

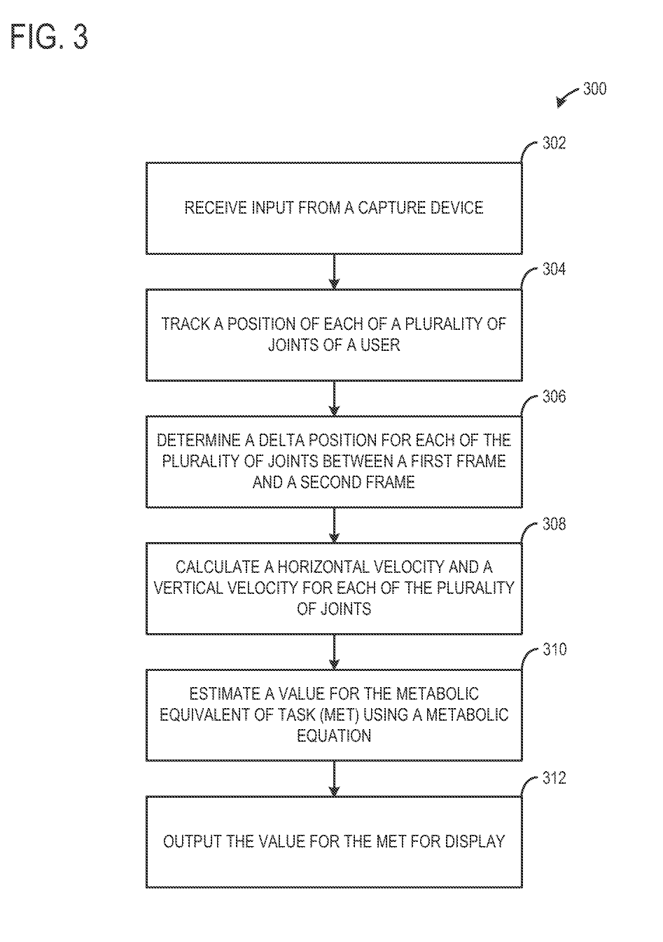

FIG. 3 is a flowchart that illustrates an example embodiment of a method 300 for estimating the MET using the gaming system of FIG. 1. Method 300 may be implemented using the hardware and software components described herein.

At 302, method 300 includes receiving input from a capture device. For example, the capture device may be depth camera 18, and the input may include a sequence of images of a user captured over time. Therefore, the sequence of images of the user may be a sequence of depth images of the user captured over time, for example.

At 304, method 300 includes tracking a position of each of a plurality of joints of the user. For example, the position of each of the plurality of joints of the user may be determined from depth information for each joint, as captured in the sequence of depth images of the user. Further, the position of each of the plurality of joints may be determined via the skeletal tracking pipeline, as described above. In this way, a three-dimensional (3D) position for each tracked joint may be determined within each frame (i.e., with each captured depth image). For example, the 3D position may be determined using a Cartesian coordinate system including x, y, and z directions.

At 306, method 300 includes determining a delta position for each of the plurality of joints between a first frame and a second frame. Delta position, as referred to herein, may be defined as a change in position. As such, the delta position may be used to determine a distance traveled by each of the plurality of joints. For example, the delta position may be based on a change in the tracked position of each of the plurality of joints between the first and second frames. Further, as referred to herein, the first frame may be a first captured image and the second frame may be a second capture image, for example. It is to be understood that the second frame may be any frame that occurs after the first frame. For example, the second frame may be a frame immediately after first frame. As another example, the second frame may be a frame that is captured a period of time after the first frame is captured. The period of time may be any suitable period of time, such as a millisecond, a second, a minute, more than one minute, or any other period of time, for example. It will be appreciated that the period of time may be a threshold period of time. For example, the threshold period of time may correspond to any of the aforementioned examples of periods of time. Further, the threshold period of time may be a period of time that is predetermined as a sufficient period of time for estimating the MET, for example. Such a threshold period of time may correspond to an elapsed period of time defined by the first and second frames. In this way, the delta distance is determined for each of the plurality of joints of the user over the elapsed period of time between the first and second frames.

At 308, method 300 includes calculating a horizontal velocity and a vertical velocity for each of the plurality of joints. For example, the horizontal velocity and the vertical velocity may be based on the delta position for each of the plurality of joints and an elapsed time between the first and second frames. For example, the horizontal velocity may be equal to a horizontal delta position for each of the plurality of joints divided by the elapsed time. As another example, the vertical velocity may be equal to a vertical delta position for each of the plurality of joints divided by the elapsed time.

Calculating the horizontal velocity may include one or more velocity components within a horizontal plane. For example, calculating the horizontal velocity may include a velocity in an x direction and a velocity in a z direction, wherein the x and z directions are from a perspective of the depth camera. As such, the x direction may represent a lateral direction from the depth camera (side-to-side), and the z direction may represent a depth direction from the depth camera (towards/away).

Similarly, calculating the vertical velocity may include one or more velocity components within a vertical plane, perpendicular to the horizontal plane. For example, calculating the vertical velocity may include a velocity in a y direction, wherein the y direction is from the perspective of the depth camera. As such, the y direction may represent an up/down direction from the depth camera.

At 310, method 300 includes estimating a value for the metabolic equivalent of task using a metabolic equation. For example, the metabolic equation may include a horizontal component and a vertical component. The horizontal and vertical components may be a summation of the horizontal velocities and vertical velocities for each of the plurality of joints, respectively. Further, the horizontal and vertical components may additionally include a horizontal variable and a vertical variable, respectively. For example, the metabolic equation may be the American College of Sports Medicine (ACSM) metabolic equation for calculating metabolic equivalent of task (MET):

$$M E T = \frac{{vo}_{2}}{3.5} \qquad Eqn 1$$

where VO₂ represents oxygen consumption, calculated by the following equation:

VO₂=Componentₕ+Componentᵥ+R Eqn 2:

where ‘R’ is a constant equal to 3.5, ‘Componentₕ’ is the horizontal component, and ‘Componentᵥ’ is the vertical component. The horizontal and vertical components may be defined by expanding Eqn 2 to the following equation:

VO₂=Kₕ(Velocityₕ)+Kᵥ(Velocityᵥ)+R Eqn 3:

where ‘Velocityₕ’ represents the horizontal velocity and ‘Velocityᵥ’ represents the vertical velocity, which may be calculated according to the delta position of the plurality of joints of the user between the first frame and the second frame and an elapsed time between the first and second frames, as described above.

Further, Eqn 3 includes ‘Kₕ’ and ‘Kᵥ’ which may represent the horizontal variable and the vertical variable, respectively. A value for ‘Kₕ’ and ‘Kᵥ’ may be determined by training the variables to reflect a broad spectrum of MET activities. For example, ‘Kₕ’ and ‘Kᵥ’ may each be an average of one or more low MET values, one or more medium MET values, and one or more high MET values. For example, a low MET value may correspond to a user interacting with gaming system 12 by sitting on a couch and watching a movie (e.g., a MET value less than 3.0). Further, a medium MET value may correspond to a user interacting with gaming system 12 by controlling a race car avatar with the user's movements in a racing game, (e.g., a MET value between 3.0 and 6.0). Further still, a high MET value may correspond to a user interacting with gaming system 12 by controlling a player avatar with the user's movements in a dancing game, (e.g., a MET value greater than 6.0). In this way, the low to high MET values may correlate with low intensity to high intensity activities, for example.

Traditional methods for estimating MET values may use a particular horizontal variable and a particular vertical variable that correspond to a specific activity. The present disclosure considers a broad spectrum of horizontal and vertical variables such that the method for estimating MET can be applied to any activity, as described herein.

It will be appreciated that the values for ‘Kₕ’ and ‘Kᵥ’ may be predetermined and analyzed from experimental data, wherein the experimental data includes values from a broad spectrum of MET values. As another example, the values for ‘Kₕ’ and ‘Kᵥ’ may be adapted for a particular user. For example, a user may be prompted to perform certain gestures, movements, activities, etc, and data from the associated skeletal tracking may be used to determine a particular ‘Kₕ’ and ‘Kᵥ’ for that user. In such a scenario, user identifying technologies may also be employed. For example, facial recognition technologies may be employed to identify the particular user such that a profile associated with that user including the user's particular ‘Kₕ’ and ‘Kᵥ’ values can be accessed to estimate MET. It will be appreciated that other user identifying technologies may be employed without departing from the scope of this disclosure.

Turning back to FIG. 3, at 312, method 300 includes outputting the value for MET for display. For example, display 16 may include a graphical user interface that displays the value for MET for the user. The value for MET may be an end value representing the value for MET following a completion of the user interaction with gaming system 12, for example. Further, the value for MET may be a real-time value representing a snap-shot and/or an accumulative value for the MET while the user is interacting with gaming system 12.

It will be appreciated that method 300 is provided by way of example and as such is not meant to be limiting. Therefore, it is to be understood that method 300 may be performed in any suitable order without departing from the scope of this disclosure. Further, method 300 may include additional and/or alternative steps than those illustrated in FIG. 3. For example, method 300 may include weighing each of the plurality of joints of the user to achieve a more accurate estimation of MET.

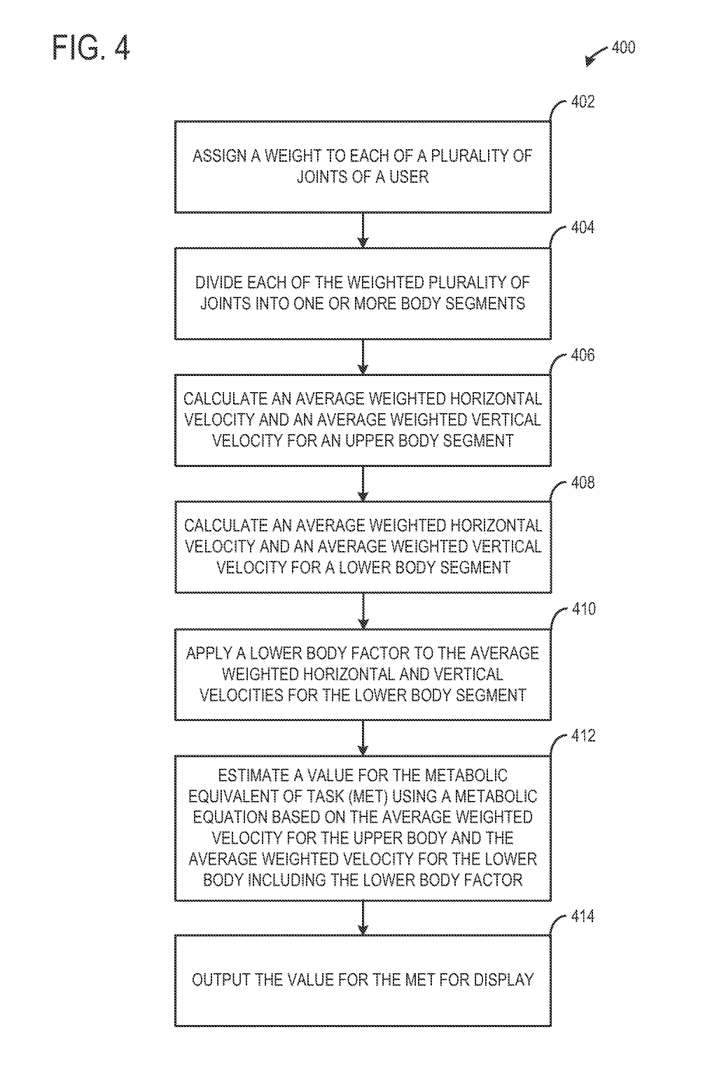

For example, FIG. 4 is a flowchart showing an example method 400 for weighing each of the plurality of joints of the user. As introduced above, weighing each of the plurality of joints of the user may result in a more accurate MET estimation than not weighing each of the plurality of joints. It will be appreciated that method 400 may include one or more steps already described with respect to FIG. 3. Further, it is to be understood that such steps may be performed similarly or with slight variations as described herein. Further, one or more steps of method 400 may transpire after determining the delta position for each of the plurality of joints between the first frame and the second frame (e.g., step 306), as described above. Method 400 may be implemented using hardware and software components described herein.

At 402, method 400 includes assigning a weight to each of a plurality of joints of a user. It will be appreciated that each joint may be assigned a particular weight. Further, it will be appreciated that a particular weight for one joint may be different than a particular weight for another joint. Each of the plurality of joints of the user may be assigned a particular weight according to virtually any weighing scheme. For example, a joint with a greater degree of freedom than another joint may be assigned a higher weighted value. As one non-limiting example, a shoulder joint may have a higher weighted value than a knee joint. Since the shoulder joint is a ball and socket type joint (rotational freedom), the shoulder joint has a greater degree of freedom than the knee joint which is similar to a hinge type joint (limited to flexion and extension).

At 404, method 400 includes dividing each of the weighted plurality of joints of the user into one or more body segments. For example, some of the weighted plurality of joints of the user may be assigned to an upper body segment. For example, the upper body segment may include one or more of the user's weighted joints between a head region and a hip region. As such, the upper body segment may include a head joint, a left hip joint, a right hip joint, and other joints anatomically positioned between the head joint and the left and right hip joints. For example, one or more joints associated with a right arm and a left arm of the user may be assigned to the upper body segment. Anatomically positioned, as used herein, may refer to a position of a joint with respect to a user's anatomy. Therefore, even though a hand joint may be physically located vertically below a hip joint (e.g., when the user bends at the hip joints to touch a foot joint), the hand joint is assigned to the upper body segment because the hand joint is anatomically positioned between the hip joints and the head joint. In other words, the hand joint is superior to the hip joints and inferior to the head joint, therefore the hand joint belongs to the upper body segment.

Similarly, other weighted plurality of joints of the user may be assigned to another body segment such as a lower body segment. For example, the lower body segment may include one or more of the user's weighted joints between the hip region and a foot region. As such, the lower body segment may include a knee joint, a foot joint, and other joints anatomically positioned between the hip region and the foot region. For example, one or more joints associated with a right leg and a left leg of the user may be assigned to the lower body segment. Therefore, even though a leg joint may be physically located vertically above a hip joint (e.g., when the user performs a high kick such as a roundhouse kick), the leg joint is assigned to the lower body segment because the leg joint is anatomically positioned between the hip joints and the foot joint. In other words, the leg joint is inferior to the hip joints and superior to the foot joint, therefore the leg joint belongs to the lower body segment.

It will be appreciated that each of the plurality of weighted joints may be assigned to only one body segment. In other words, a single joint may not be assigned to more than one body segment. In this way, each of the weighted plurality of joints of the user may be analyzed without duplicating a particular weighted joint in two body segments. Further, since the hip region is described above as a divider between the upper body segment and the lower body segment, it will be appreciated that one or more of the hips joints may be assigned to the upper body segment or the lower body segment. For example, both the left hip joint and the right hip joint may be assigned to the upper body segment or both the left hip joint and the right hip joint may be assigned to the lower body segment. Alternatively, one hip joint may be assigned to the upper body segment and the other hip joint may be assigned to the lower body segment.

Turning back to FIG. 4, at 406, method 400 includes calculating an average weighted horizontal velocity and an average weighted vertical velocity for the upper body segment. For example, the average weighted horizontal and vertical velocities for the upper body segment may be calculated by determining a delta position for each of the weighted plurality of joints within the upper body region between a first frame and a second frame, and an elapsed time between the first frame and the second frame, similar to the above description. For example, the average weighted velocities for the upper body segment may be calculated according to equation 4 and equation 5 provided below. It will be appreciated that equations 4 and 5 are provided as non-limiting examples.

$$\begin{matrix}U B {Velocity}_{h} = \frac{{\sum }_{i = hip}^{Head} [ {Velocity}_{h} ( i ) Weight ( i ) ]}{Total Weight} & Eqn 4 \\ U B {Velocity}_{v} = \frac{{\sum }_{i = hip}^{Head} [ {Velocity}_{v} ( i ) Weight ( i ) ]}{Total Weight} & Eqn 5\end{matrix}$$

As shown in equations 4 and 5, ‘UB’ indicates the upper body segment and the index ‘i’ represents a specific joint. Further, the total weight may be the sum of the weights applied to each of the plurality of joints assigned to the upper body segment, for example.

At 408, method 400 includes calculating an average weighted horizontal velocity and an average weighted vertical velocity for the lower body segment. For example, the average weighted horizontal and vertical velocities for the lower body segment may be calculated by determining a delta position for each of the weighted plurality of joints within the lower body region between the first frame and the second frame, and an elapsed time between the first frame and the second frame, similar to the above description. For example, the average weighted velocities for the lower body segment may be calculated according to equation 6 and equation 7 provided below. It will be appreciated that equations 6 and 7 are provided as non-limiting examples.

$$\begin{matrix}L B {Velocity}_{h} = \frac{{\sum }_{i = foot}^{Hip - 1} [ {Velocity}_{h} ( i ) Weight ( i ) ]}{Total Weight} & Eqn 6 \\ L B {Velocity}_{v} = \frac{{\sum }_{i = foot}^{Hip - 1} [ {Velocity}_{v} ( i ) Weight ( i ) ]}{Total Weight} & Eqn 7\end{matrix}$$

As shown in equations 6 and 7, ‘LB’ indicates the lower body segment and the index ‘i’ represents a specific joint. Further, the total weight may be the sum of the weights applied to each of the plurality of joints assigned to the lower body segment, for example.

At 410, method 400 includes applying a lower body factor to the average weighted horizontal and vertical velocities for the lower body segment. For example, the lower body segment and the upper body segment may have different effects on the MET. Therefore, the lower body factor may be applied to the average weighted horizontal and vertical velocities for the lower body segment to account for this difference in affect on the MET.

For example, the lower body segment may have a greater affect on the MET because the lower body segment carries the weight of the upper body segment. Additionally and/or alternatively, the lower body segment may have a greater affect on the MET because the lower body experiences frictional forces with the ground during activity. In this way, even though joints within the lower body segment and the upper body segment may have similar velocities, the affect of joints within the lower body segment may affect the MET value more than the joints within the upper body segment, for example. The inventors herein have recognized that a lower body factor between a value of 2 and a value of 3 accounts for this difference in affect. However it will be appreciated that other lower body factors are possible and/or an upper body factor may be applied to the upper body segment velocities without departing from the scope of this disclosure.

At 412, method 400 includes estimating a value for the metabolic equivalent of task (MET) using a metabolic equation. For example, the metabolic equation may be based on the average weighted velocity for the upper body and the average weighted velocity for the lower body wherein the average weighted velocity for the lower body includes the applied lower body factor. For example, the MET may be calculated according to equation 1 as described above, and further, a value for the oxygen consumption (VO₂) may be determined by using equations 8, 9, and 10 as provided below. It will be appreciated that equations 8, 9 and 10 are provided as non-limiting examples.

BodyVelocityₕ=UBVelocityₕ+LBFactor×LBVelocityₕ Eqn 8:

BodyVelocityᵥ=UBVelocityᵥ+LBFactor×LBVelocityᵥ Eqn 9:

VO₂=Kₕ(BodyVelocityₕ)+Kᵥ(BodyVelocityᵥ)+R Eqn 10:

As shown in equations 8 and 9, ‘UB’ indicates the upper body segment and ‘LB’ indicates the lower body segment. Further, it will be appreciated that equations 8, 9, and 10 include variables that are similar to variables included in some of the previously described equations and for the sake of brevity will not be described further.

At 414, method 400 includes outputting the calculated MET value for display. For example, display 16 may include a graphical user interface that displays the value for MET for the user. The value for MET may be an end value, a real-time value, a snap-shot value and/or an accumulative value for the MET as described above.

It will be appreciated that method 400 is provided by way of example and as such is not meant to be limiting. Therefore, it is to be understood that method 400 may be performed in any suitable order without departing from the scope of this disclosure. Further, method 400 may include additional and/or alternative steps than those illustrated in FIG. 4. For example, method 400 may include a calculation for a caloric burn based on the calculated MET value. Further, the calculated MET value may be used to determine other physical parameters that may assess an aspect of the user's physical performance when interacting with the computing system.

As another example, method 400 may include tuning the weighted factors for a particular user. In some embodiments, tuning the weighted factors for a particular user may include a user identifying technology. For example, a user may be identified by facial recognition technology, and/or by another user identifying technology.

In this way, the value for MET can be estimated for a user interacting with a computing device, such as gaming system 12. Further, since the movements (or lack thereof) of a user are tracked, estimating the value for MET may be accomplished more accurately without assuming the particular activity that the user is actually performing.

In some embodiments, the above described methods and processes may be tied to a computing system including one or more computers. In particular, the methods and processes described herein may be implemented as a computer application, computer service, computer API, computer library, and/or other computer program product.

FIG. 5 schematically shows a non-limiting computing system 70 that may perform one or more of the above described methods and processes. Computing system 70 is shown in simplified form. It is to be understood that virtually any computer architecture may be used without departing from the scope of this disclosure. In different embodiments, computing system 70 may take the form of a mainframe computer, server computer, desktop computer, laptop computer, tablet computer, home entertainment computer, network computing device, mobile computing device, mobile communication device, gaming device, etc.

Computing system 70 includes a processor 72 and a memory 74. Computing system 70 may optionally include a display subsystem 76, communication subsystem 78, sensor subsystem 80 and/or other components not shown in FIG. 5. Computing system 70 may also optionally include user input devices such as keyboards, mice, game controllers, cameras, microphones, and/or touch screens, for example.

Processor 72 may include one or more physical devices configured to execute one or more instructions. For example, the processor may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The processor may include one or more processors that are configured to execute software instructions. Additionally or alternatively, the processor may include one or more hardware or firmware logic machines configured to execute hardware or firmware instructions. Processors of the processor may be single core or multicore, and the programs executed thereon may be configured for parallel or distributed processing. The processor may optionally include individual components that are distributed throughout two or more devices, which may be remotely located and/or configured for coordinated processing. One or more aspects of the processor may be virtualized and executed by remotely accessible networked computing devices configured in a cloud computing configuration.

Memory 74 may include one or more physical, non-transitory, devices configured to hold data and/or instructions executable by the processor to implement the herein described methods and processes. When such methods and processes are implemented, the state of memory 74 may be transformed (e.g., to hold different data).

Memory 74 may include removable media and/or built-in devices. Memory 74 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Memory 74 may include devices with one or more of the following characteristics: volatile, nonvolatile, dynamic, static, read/write, read-only, random access, sequential access, location addressable, file addressable, and content addressable. In some embodiments, processor 72 and memory 74 may be integrated into one or more common devices, such as an application specific integrated circuit or a system on a chip.

FIG. 5 also shows an aspect of the memory in the form of removable computer-readable storage media 82, which may be used to store and/or transfer data and/or instructions executable to implement the herein described methods and processes. Removable computer-readable storage media 82 may take the form of CDs, DVDs, HD-DVDs, Blu-Ray Discs, EEPROMs, and/or floppy disks, among others.

It is to be appreciated that memory 74 includes one or more physical, non-transitory devices. In contrast, in some embodiments aspects of the instructions described herein may be propagated in a transitory fashion by a pure signal (e.g., an electromagnetic signal, an optical signal, etc.) that is not held by a physical device for at least a finite duration. Furthermore, data and/or other forms of information pertaining to the present disclosure may be propagated by a pure signal.

The terms “module,” “program,” and “engine” may be used to describe an aspect of computing system 70 that is implemented to perform one or more particular functions. In some cases, such a module, program, or engine may be instantiated via processor 72 executing instructions held by memory 74. It is to be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” are meant to encompass individual or groups of executable files, data files, libraries, drivers, scripts, database records, etc.

It is to be appreciated that a “service”, as used herein, may be an application program executable across multiple user sessions and available to one or more system components, programs, and/or other services. In some implementations, a service may run on a server responsive to a request from a client.

When included, display subsystem 76 may be used to present a visual representation of data held by memory 74. As the herein described methods and processes change the data held by the memory, and thus transform the state of the memory, the state of display subsystem 76 may likewise be transformed to visually represent changes in the underlying data. Display subsystem 76 may include one or more display devices utilizing virtually any type of technology. Such display devices may be combined with processor 72 and/or memory 74 in a shared enclosure, or such display devices may be peripheral display devices.

When included, communication subsystem 78 may be configured to communicatively couple computing system 70 with one or more other computing devices. Communication subsystem 78 may include wired and/or wireless communication devices compatible with one or more different communication protocols. As nonlimiting examples, the communication subsystem may be configured for communication via a wireless telephone network, a wireless local area network, a wired local area network, a wireless wide area network, a wired wide area network, etc. In some embodiments, the communication subsystem may allow computing system 70 to send and/or receive messages to and/or from other devices via a network such as the Internet.

Sensor subsystem 80 may include one or more sensors configured to sense one or more human subjects, as described above. For example, the sensor subsystem 80 may comprise one or more image sensors, motion sensors such as accelerometers, touch pads, touch screens, and/or any other suitable sensors. Therefore, sensor subsystem 80 may be configured to provide observation information to processor 72, for example. As described above, observation information such as image data, motion sensor data, and/or any other suitable sensor data may be used to perform such tasks as determining a position of each of a plurality of joints of one or more human subjects.

In some embodiments, sensor subsystem 80 may include a depth camera 84 (e.g., depth camera 18 of FIG. 1). Depth camera 84 may include left and right cameras of a stereoscopic vision system, for example. Time-resolved images from both cameras may be registered to each other and combined to yield depth-resolved video.

In other embodiments, depth camera 84 may be a structured light depth camera configured to project a structured infrared illumination comprising numerous, discrete features (e.g., lines or dots). Depth camera 84 may be configured to image the structured illumination reflected from a scene onto which the structured illumination is projected. Based on the spacings between adjacent features in the various regions of the imaged scene, a depth image of the scene may be constructed.

In other embodiments, depth camera 84 may be a time-of-flight camera configured to project a pulsed infrared illumination onto the scene. The depth camera may include two cameras configured to detect the pulsed illumination reflected from the scene. Both cameras may include an electronic shutter synchronized to the pulsed illumination, but the integration times for the cameras may differ, such that a pixel-resolved time-of-flight of the pulsed illumination, from the source to the scene and then to the cameras, is discernable from the relative amounts of light received in corresponding pixels of the two cameras.

In some embodiments, sensor subsystem 80 may include a visible light camera 86. Virtually any type of digital camera technology may be used without departing from the scope of this disclosure. As a non-limiting example, visible light camera 86 may include a charge coupled device image sensor.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A method for estimating a metabolic equivalent of task for use with a computing device, the method comprising:
receiving input from a capture device including a sequence of images of a user captured over time;
tracking a position of each of a plurality of joints of the user from the sequence of images;
determining a distance for each of the plurality of joints between a first frame and a second frame based on a change in position for each of the tracked plurality of joints between the first and second frames;
calculating a horizontal velocity and a vertical velocity for each of the plurality of joints based on the distance for each of the plurality of joints and an elapsed time between the first and second frames;
estimating a value for the metabolic equivalent of task using a metabolic equation, the metabolic equation including a horizontal component and a vertical component, the horizontal and vertical components based on the calculated horizontal and vertical velocities for each of the plurality of joints; and
outputting the value for display.

2. The method of claim 1, further comprising weighing each of the plurality of joints according to a weighing scheme.

3. The method of claim 1, wherein the capture device is a depth camera and wherein the sequence of images is a sequence of depth images.

4. The method of claim 1, further comprising training the computing device to recognize different users and adjusting the value for a particular user.

5. The method of claim 1, wherein the metabolic equation includes a value for an oxygen consumption.

6. The method of claim 5, wherein the oxygen consumption includes a horizontal variable and a vertical variable, the horizontal and vertical variables based on a broad spectrum of metabolic equivalent values.

7. The method of claim 1, wherein the horizontal velocity includes a velocity in an x direction and a velocity in a z direction.

8. The method of claim 1, wherein the vertical velocity includes a velocity in a y direction.

9. A computing device including a memory holding instructions executable by a processor to:
capture a plurality of images of a user using a depth camera associated with the computing device;
track a position for each of a plurality of joints of the user over time;
determine a change in position for each of the plurality of joints between a first frame and a second, successive, frame, the change in position determined from the tracked position for each of the plurality of joints;
calculate a velocity for each of the plurality of joints based on the change in position over an elapsed time between the first and second frames; and
output a value for a metabolic equivalent of task, the value outputted from a metabolic equation including a horizontal velocity component and a vertical velocity component for each of the plurality of joints.

10. The device of claim 9, wherein the outputted value is output on a display of the computing device.

11. The device of claim 9, wherein the user is tracked for a threshold period of time.

12. The device of claim 11, wherein the value is a total value for the threshold period of time, wherein the total value is a sum of the metabolic equivalent of task calculated between each frame and a successive frame within the threshold period of time.

13. The device of claim 9, further comprising instructions to weigh each of the plurality of joints according to a weighing scheme.

14. The device of claim 13, wherein the weighing scheme includes assigning each of the plurality of joints to an upper body segment or a lower body segment.

15. The device of claim 14, wherein the lower body segment has a higher weighted value than the upper body segment.

16. The device of claim 15, wherein the metabolic equation is
$$M E T = \frac{{vo}_{2}}{3.5} ,$$
wherein VO₂ is a variable for oxygen consumption.

17. The device of claim 15, wherein oxygen consumption is calculated using an oxygen consumption equation.

18. The device of claim 17, wherein the oxygen consumption equation is VO₂=Kₕ(BodyVelocityₕ)+Kᵥ(BodyVelocityᵥ)+3.5.

19. The device of claim 9, wherein the computing device is a gaming device.

20. A computing device including a memory holding instructions executable by a processor to:
capture a plurality of images of a user using a depth camera associated with the computing device;
track a position for each of a plurality of joints of the user over time;
determine a change in position for each of the plurality of joints between a first frame and a second frame, the change in position determined from the tracked position for each of the plurality of joints;
calculate a velocity for each of the plurality of joints based on the change in position over an elapsed time between the first and second frames;
weigh each of the plurality of joints according to a weighing scheme, the weighing scheme including an upper body segment and a lower body segment, the upper body segment weighed differently from the lower body segment, and
output a value for a metabolic equivalent of task, the value outputted from a metabolic equation including a horizontal velocity component and a vertical velocity component for each of the weighed plurality of joints.
